(19) United States
(12) Patent Application Publication
(10) Pub. No.: US 2025/0339813 A1
(43) Pub. Date: Nov. 6, 2025

(54) ABSORBENT FOR THE QUANTITATIVE REMOVAL OF CARBON DIOXIDE FROM A GAS STREAM AND USE THEREOF

(71) Applicant: Elementar Analysensysteme GmbH, Langenselbold (DE)

(72) Inventors: Jan HARTWIG, Alzenau (DE); Sabine KRAUS, Alzenau (DE)

(21) Appl. No.: 18/865,415

(22) PCT Filed: Jun. 16, 2023
(86) PCT No.: PCT/EP2023/066272

§ 371 Date: Jul. 23, 2025

(30) Foreign Application Priority Data
Jun. 30, 2022 (DE) 10 2022 116 394.6

(51) Int. Cl. B01D 53/62; B01D 53/81; B01J 20/18; B01J 20/28

(52) U.S. Cl. CPC B01D 53/62; B01D 53/81; B01J 20/18; B01J 20/2808; B01D 2251/304; B01D 2251/404; B01D 2251/604; B01D 2253/108; B01D 2257/504

(57) ABSTRACT

The invention relates to a material for the quantitative removal of carbon dioxide from a gas stream. The material comprises a mixture of sodium hydroxide, calcium hydroxide and at least one drying agent, wherein the drying agent is a molecular sieve. The invention also extends to the use of this material in a suitable apparatus such as a gas analysis system.

The invention relates to a mixture for the quantitative removal of carbon dioxide from a gas stream.

In the analysis of gas streams, it is necessary to remove carbon dioxide (CO2) quantitatively, the term quantitative in the sense of the invention being understood to mean that the carbon dioxide content is reduced to a value of less than 1 ppm. This relates in particular to the analysis of gas streams by means of a thermal conductivity detector, IR spectroscopy or a mass spectrometer. The gas streams to be analysed are therefore usually purified, whereby an absorbent is usually provided in a tube and the gas stream is passed through this tube.

Until now, the use of sodium hydroxide (NaOH) as an absorbent was generally known, which proceeds according to the following reaction equation:

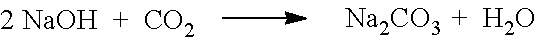

Sodium hydroxide has sufficient reactivity with carbon dioxide to actually ensure quantitative removal. A disadvantage in the analysis, however, is the water released in the process, which must be removed for correct measurements in the downstream analysis.

However, the sodium hydroxide cannot be used in its pure form, as this would result in the formation of sodium carbonate (NaCO3). This sodium carbonate would form as solid agglomerates and thus adversely change the flow profile of the gas stream or, in the worst case, lead to complete blockage.

A further disadvantage when used in the analysis of gas streams is that the sodium carbonate primarily forms on the surface of the bulk, while sodium hydroxide remains inside the material. When rinsing with water, both the carbonate and the hydroxide dissolve, resulting in a strongly alkaline and therefore hazardous solution.

In the past, sodium hydroxide was therefore applied to a carrier material, preferably a silicate carrier. In most cases, quartz glass chips were also applied to this carrier material, preferably in a ratio of 1:1 to sodium hydroxide, in order to mechanically prevent the formation of larger sodium carbonate agglomerates. However, the production of such a material is complex. In addition, blocking with the disadvantages described above cannot be reliably and completely avoided in all possible applications.

The use of so-called soda lime, a mixture of sodium hydroxide and calcium hydroxide, is also known from the state of the art. In addition to carbon dioxide removal in elemental analysis, this mixture, also known as soda lime, is used in rebreathers to bind the exhaled carbon dioxide.

The calcium hydroxide (Ca(OH)₂) contained does not have sufficient reactivity on its own to remove the required amount of carbon dioxide from the gas flow and is therefore not suitable as an absorbent for the quantitative removal of carbon dioxide. In the mixture of sodium hydroxide and calcium hydroxide, sodium hydroxide reacts with carbon dioxide on the surface in a known manner in a first reaction. In a second reaction, the sodium carbonate resulting from the reaction of sodium hydroxide with carbon dioxide reacts with the calcium hydroxide. This reaction produces calcium carbonate. At the same time, the sodium hydroxide is regenerated, which greatly increases the capacity of the absorbent. The gross reaction equations are as follows:

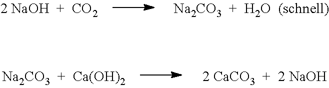

However, these reactions are strongly water-dependent, which can be partly explained by the fact that the chemical equilibrium reactions that lead to the formation of carbonate and hydrogen carbonate ions, which are necessary intermediate stages in the two reactions, only occur in the presence of water.

However, gas streams to be analysed are usually already pre-purified so that they are not saturated with water and would therefore discharge this excess water when flowing through the material. However, this is disadvantageous for a number of reasons: If the stream containing water actually reaches the detector, it would falsify its signal. Alternatively, the gas stream would have to be dried after CO₂ absorption, which would entail a high consumption of drying agent. In addition, the discharge of water from the system would have a negative effect on the equilibrium reactions taking place, so that the second reaction with calcium hydroxide could hardly take place and within a very short time the situation would be comparable to the use of pure sodium hydroxide.

It is therefore the task of the invention to provide a simple and safe material for the quantitative removal of carbon dioxide from a gas stream for downstream gas analysis, in particular elemental analysis. This also includes the fact that the composition of the gas stream, in particular with regard to the water content, must not be changed in such a way that the downstream desiccant is quickly consumed and/or the signal of the detector would be distorted.

This problem is solved with a material according to claim 1.

One such material comprises a mixture of sodium hydroxide, calcium hydroxide and at least one desiccant. The additional use of a desiccant ensures that the gas stream to be analyzed does not remove large quantities of water from the absorption of the carbon dioxide. However, it should be noted that most desiccants are not PH neutral. As an example of a classic desiccant, Sicapent® is a typical acidic representative, which would therefore react with sodium or calcium hydroxide and is therefore unsuitable for use as a mixing component.

In addition, the desiccant must not remove the water so permanently that it is no longer available for the reaction equilibrium. Surprisingly, it was found that the use of molecular sieve, also known as molecular sieve, fulfils these requirements.

A molecular sieve does not react with sodium hydroxide or calcium hydroxide. At the same time, it reliably retains the water in the mixture according to the invention and thus does not additionally influence the elemental analysis or downstream stages such as drying. At the same time, however, the molecular sieve does not remove the water from the mixture so completely that the regeneration of the sodium hydroxide by the calcium hydroxide is prevented, as it is still present within the pores with an equilibrium effect. The simultaneous fulfilment of all three requirements by the desiccant used makes it possible to use sodium hydroxide and calcium hydroxide for the quantitative removal of carbon dioxide.

The positive effect of the indirect provision of water in the molecular sieve is so great that the service life of the material according to the invention is longer than when an identical quantity of sodium hydroxide and calcium hydroxide is used without the use of a drying agent, disregarding the negative effects of discharged water.

For the purposes of the invention, the term molecular sieve is to be understood as a functional designation for natural and synthetic zeolites and other substances which have a high adsorption capacity for gases, vapours and dissolved substances with certain molecular sizes. In particular, the invention relates to natural and synthetic zeolites. Such molecular sieves have a comparably large internal surface area, preferably in the range of 500-760 m²/g, and have a very homogeneous pore diameter, which is in the order of magnitude of the diameter of molecules. The pore diameter given in Ångström is generally used to classify molecular sieves.

Preferably, due to the influence of water on the reactions already described, the mixture of sodium hydroxide, calcium hydroxide and a drying agent used also comprises water from the outset and thus the water content during operation is higher than that produced by the reaction of the sodium hydroxide with the carbon dioxide.

According to the invention, the mixture comprises 0.5 to 5% by weight of sodium hydroxide, 20 to 70% by weight of calcium hydroxide and 25 to 79.5% by weight of drying agent, preferably 1 to 3% by weight of sodium hydroxide, 39 to 59% by weight of calcium hydroxide and 38 to 62% by weight of drying agent, particularly preferably 1.5 to 3% by weight of sodium hydroxide, 39 to 59% by weight of calcium hydroxide and 38 to 59.5% by weight of drying agent. % desiccant, with additional pre-saturation with water, so that 1 to 3% by weight of sodium hydroxide, 33 to 55% by weight of calcium hydroxide, 3 to 10% by weight of water and 32 to 63% by weight of desiccant, preferably 1.5 to 3.0% by weight of sodium hydroxide, 35 to 50% by weight of calcium hydroxide, 4 to 10% by weight of water and 37 to 59.5% by weight of desiccant. The relatively small amount of sodium hydroxide reliably prevents the local formation of sodium carbonate and the associated blockages of the gas flow. At the same time, the amount of calcium hydroxide ensures a long service life of the absorbent. The amount of desiccant and, if necessary, the pre-saturation with water is adjusted in such a way that water breakthrough is reliably avoided and at the same time the conversion of the calcium hydroxide is not limited by the amount of water present.

It has also been found to be favorable if the mixture consists of at least 90% by weight, preferably 95% by weight, of sodium hydroxide, calcium hydroxide, at least one drying agent and water, so that other components do not influence the reaction.

In a preferred embodiment, however, the material contains an indicator for carbon dioxide and/or an indicator for water. This has the advantage that when the material is used, for example when filling a bulk, any storage faults that have led to exhaustion of the absorption capacities before use are visible. Similarly, the material can also be used in a container with an at least partially transparent casing so that the end of the service life can also be detected visually.

In a particularly simple embodiment, this is a pH indicator which, when the calcium hydroxide present is exhausted and the second gross reaction therefore no longer takes place, indicates the resulting pH change.

The drying agent can be used in any form, whereby a granular form, in particular a spherical or rod-shaped design, has proved to be particularly favorable for a uniform flow profile of the gas stream.

The invention also includes a device for absorbing carbon dioxide. Such a device has a gas-tight housing with a gas inlet and a gas outlet. The volume defined by this housing is at least partially filled with the material according to the invention according to one of claims 1 to 4. A particularly simple embodiment of such a device is a filled tubular reactor.

The device can be made of any material that is inert to the gas stream to be analyzed and is gastight. Metals, glass and plastic are particularly conceivable. Glass and, in some cases, plastic have the advantage of being transparent, while plastic is particularly suitable due to its break resistance and, in some cases, low manufacturing costs.

Finally, the invention is also directed to the use of the material according to the invention according to one of claims 1 to 5 and/or a device according to the invention according to claim 5 for the quantitative removal of carbon dioxide in a gas analysis system, in particular in elemental analysis and/or IRMS. As described, the material according to the invention is particularly suitable here because, in addition to quantitative removal, it must also be ensured that the gas stream freed of carbon dioxide has not been changed by other components such as water or that its flow behavior has not been changed.

Further embodiments of the invention are shown in the example and the figures with their associated description. Each feature is to be regarded as disclosed separately and in any combination. Some of the figures are slightly simplified and schematic.

It shows:

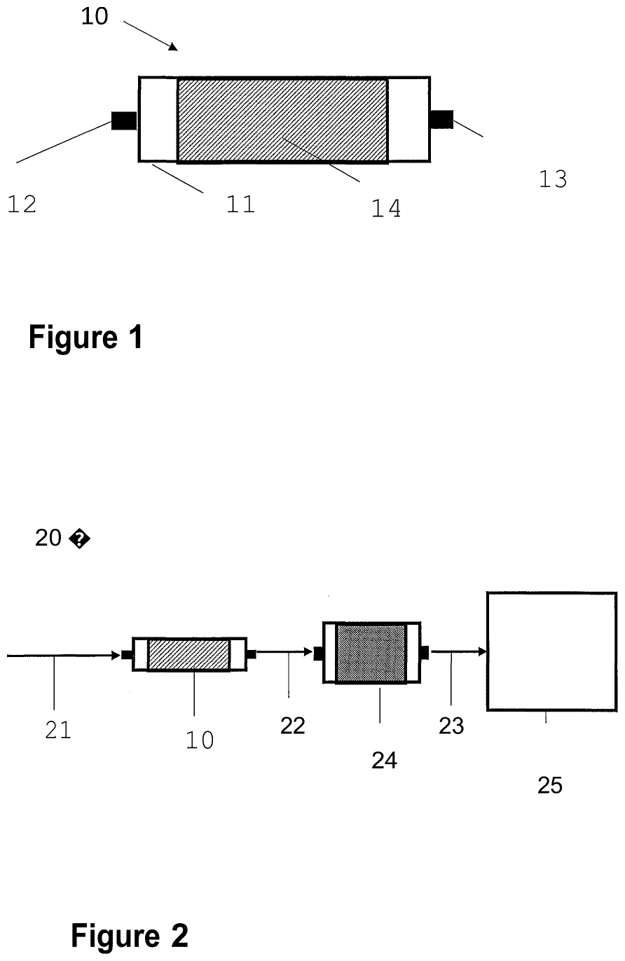

FIG. 1: a schematic embodiment of a device according to the invention and

FIG. 2: a schematic diagram of the use of a device according to the invention in a gas analysis system

FIG. 1 shows the device 10 according to the invention, which is designed as a preferably cylindrical housing 11 with a gas inlet 12 and a gas outlet 13. Inside there is a filling 14 with the material according to the invention, which is inserted in such a way that the gas flow flowing in through the gas inlet 12 must always pass through the material before it can escape via the gas outlet 13.

FIG. 2 shows a very simplified gas analysis system 20, preferably an elemental analysis system, in which the material according to the invention is used for the quantitative removal of carbon dioxide. Via lines 21, 22 and 23, the gas flow is first passed through the device 10 according to the invention and then through a drying device 24 before it is analyzed in the detector 25.

As an alternative to the graphical representation, the functions of the devices 10 and 24, i.e. carbon dioxide removal and drying, can also be arranged in a device in such a way that this device has two segments into which a corresponding material is filled. In particular, this may be a tube, a first part of which is filled with the mixture according to the invention and a second part of which is filled with a drying agent downstream with respect to the gas flow passing through.

In addition, a pre-drying process not shown can be provided before the removal of carbon dioxide.

### EXAMPLE

The following example shows how many measurements can be carried out with which type of absorbent for the quantitative removal of carbon dioxide before a signal originating from the carbon dioxide that is no longer completely absorbed and/or water that is no longer completely removed by the downstream drying device is recorded in the detector. The values given are average values over 5 service life tests in each case:

| absorbent material | Number of measurements |
|---|---|
| sodium hydroxide on carrier material | 300 |
| sodium hydroxide/calcium hydroxide | 30 |
| (section S1) with subsequent drying | |
| via a section S2 (S1 = S2) | |
| sodium hydroxide/calcium hydroxide | 60 |
| (section S1) with subsequent drying | |
| via a section S2 through which a flow | |
| must pass, where S2 = 2*S1 | |
| mixture according to the invention | 500 |

It has been shown that the material according to the invention is superior to all other absorbent materials in terms of service life. In addition, the subsequent drying section and the associated pressure losses that occur when using the mixture according to the invention can be designed to be comparatively short.

### LIST OF REFERENCES

10 device
11 housing
12 Gas inlet
13 Gas outlet
14 filling with material according to the invention
20 gas analysis system
21-23 line
24 Drying device
25 detector

1. Material for the quantitative removal of carbon dioxide from a gas stream, the material containing a mixture of sodium hydroxide, calcium hydroxide and at least one drying agent, and the drying agent being a molecular sieve, characterized in that the mixture comprises 1 to 3% by weight of sodium hydroxide, 33 to 55% by weight of calcium hydroxide, 3 to 10% by weight of water and 32 to 63% by weight of drying agent.

2. Material for the quantitative removal of carbon dioxide from a gas stream according to claim 1, characterized in that at least 70% by weight of the molecular sieve has a pore size of 3 to 5 Å.

3. Material for the quantitative removal of carbon dioxide from a gas stream according to claim 1, characterized in that the mixture consists of at least 90% by weight of sodium hydroxide, calcium hydroxide, at least one drying agent and water.

4. Material for the quantitative removal of carbon dioxide from a gas stream according to claim 1, characterized in that the material contains an indicator for carbon dioxide and/or an indicator for water.

5. Device (10) for absorbing carbon dioxide, having a gas-tight housing (11) which has a gas inlet (12) and a gas outlet (13) and whose volume is at least partially filled with a material according to claim 1.

6. Use of a material according to any claim 1 for the quantitative removal of carbon dioxide in an analytical system which operates by means of gas analysis (20).

7. Use of a material for the quantitative removal of carbon dioxide from a gas stream, the material containing a mixture of sodium hydroxide, calcium hydroxide and at least one drying agent, and the drying agent being a molecular sieve, characterized in that the mixture comprises 1 to 3% by weight of sodium hydroxide, 33 to 55% by weight of calcium hydroxide, 3 to 10% by weight of water and 32 to 63% by weight of drying agent and a device according to claim 5 for the quantitative removal of carbon dioxide in an analytical system which operates by means of gas analysis (20).
